(19) United States
(12) Patent Application Publication
(10) Pub. No.: US 2017/0056253 A1
(43) Pub. Date: Mar. 2, 2017

(54) Absorbent Article Having A High Content Of Bio-Based Materials

(71) Applicant: Fitesa Nonwoven, Inc., Simpsonville, SC (US)

(72) Inventors: Stephen O. Chester, Simpsonville, SC (US); Gary Drews, Greenville, SC (US); Daniel Kong, Simpsonville, SC (US); David Dudley Newkirk, Greer, SC (US)

(21) Appl. No.: 14/839,026

(22) Filed: Aug. 28, 2015

(51) Int. Cl. A61F 13/15; A61F 13/56; A61F 13/49; A61F 13/472; A61F 13/515; A61F 13/514

(52) U.S. Cl. CPC A61F 13/15252; A61F 13/515; A61F 13/51478; A61F 13/51401; A61F 13/49; A61F 13/472; A61F 13/5644; A61F 2013/51028

(57) ABSTRACT

Bio-based absorbent articles are provided. Bio-based absorbent articles include a topsheet, a backsheet, and an absorbent core sandwiched there between. The topsheet and backsheet are attached to each other along opposing surfaces to define a cavity in which the absorbent core is enclosed. The components of absorbent article are selected so that at least 75% of the material comprising the absorbent article comprises a bio-based material, and preferably at least 80%, 85%, 90%, and 95% of the material comprising the absorbent article comprises a bio-based material.

### FIELD

The present invention relates generally to absorbent articles, and more particularly to absorbent articles having a high bio-based content.

### BACKGROUND

Disposable absorbent garments have numerous applications including diapers, training pants, feminine care products, and adult incontinence products. The typical disposable absorbent garment is formed as a composite structure including an absorbent assembly disposed between a liquid permeable bodyside liner and a liquid impermeable outer cover. These components can be combined with other materials and features such as elastic materials and containment structures to form a product that is specifically suited to its intended purposes.

For example, wearable absorbent articles, such as diapers and adult incontinence products, typically include a containment assembly having a liquid impervious backsheet, a liquid pervious topsheet, and a core comprising an absorbent material disposed between the topsheet and backsheet. In some applications, the absorbent article may also include an elastic waist feature that allows the absorbent article to move and conform to the waist of the wearer as the wearer sits, stands, or moves. The absorbent article may also include elastic cuffs that extend about and conform to the legs of the wearer.

Traditionally, many materials used in the production of such absorbent articles are prepared from thermoplastic polymers, such as polyester, polystyrene, polyethylene, and polypropylene. These polymers are generally very stable and can remain in the environment for a long time. Recently, however, there has been a trend to develop articles and products that are considered environmentally friendly and sustainable. As part of this trend, there has been a desire to produce ecologically friendly products comprised of increased sustainable content in order to reduce the content of petroleum based materials.

Accordingly, there still exists a need for absorbent articles prepared from bio-based materials.

### SUMMARY

In one embodiment, embodiments of the invention are directed to bio-based absorbent articles having a bio-based material content of at least 75 weight percent, based on total weight of the absorbent article. In one embodiment, the bio-based absorbent article comprises a topsheet, a backsheet, and an absorbent core disposed there between, wherein the absorbent article has a bio-based material content of at least 75 weight percent, based on total weight of the absorbent article. Preferably, the bio-based material content is at least 80%, 85%, 90%, or 95% by weight of the absorbent article.

In one embodiment, one or more of the topsheet and backsheet comprises a nonwoven web comprising bicomponent fibers in which the fibers have a sheath-core configuration. In a preferred embodiment, the fibers having a core of polylactic acid (PLA), and a sheath comprising a bio-based derived polyethylene polymer, such as sugar cane derived polyethylene. In another embodiment, the fibers having a core of PLA, and a sheath comprising a polypropylene polymer. In still a further embodiment, the bio-based absorbent article comprises fibers having a core of a lignin based polymer and a sheath comprising a bio-based derived polyethylene.

In one embodiment, the bio-based absorbent article comprises fibers having a core of PLA, and a sheath comprising PLA, wherein the core has a melting temperature that is higher than the melting temperature of the PLA polymer comprising the sheath. In particular, in one embodiment, the core comprises a PLA having a lower d-enantiomer content than that of the PLA comprising the sheath.

Preferably, the topsheet and backsheet are adhesively joined to each other with a bio-based adhesive.

In a preferred embodiment, the absorbent core comprises an acrylic polymer that is derived from the conversion of a bio-based 3-hydroxypropionic acid (3-HP) to glacial acrylic acid. In some embodiments, the core may comprise a liquid acquisition system in which a bio-based acquisition layer is positioned between the core and the top sheet.

In one embodiment, the backsheet comprises a laminate comprising a film layer of a sugar cane derived polyethylene polymer that is adhesively laminated to nonwoven web comprising bicomponent fibers in which the fibers have a sheath-core configuration. In a preferred embodiment, the bicomponent fibers of the nonwoven web comprise 1) a core comprising PLA, and a sheath comprising a bio-based derived polyethylene polymer, 2) fibers comprising a core of polylactic acid PLA, and a sheath comprising a polypropylene polymer, 3) fibers having a core of a lignin based polymer and a sheath comprising bio-based derived polyethylene, or 4) fibers comprising a core of PLA, and a sheath comprising PLA, wherein the core has a melting temperature that is higher than the melting temperature of the PLA polymer comprising the sheath.

In one embodiment, the bio-based absorbent article is in the form of a diaper. In other embodiments, the bio-based absorbent article is in the form of a feminine sanitary pad.

In a preferred embodiment, the present invention provides a bio-based absorbent article, comprising a core region having an absorbent core and a chassis region surrounding the core region, said chassis region comprising front, back and waist regions, while the core region is located at least in a crotch portion of the article, a liquid impermeable backsheet is arranged at least in the core region on the garment-facing side of the absorbent core and a liquid permeable topsheet is arranged at least in the core region on the wearer-facing side of the absorbent core, wherein the absorbent article has a bio-based material content of at least 75 weight percent, based on total weight of the absorbent article.

In some embodiments, the bio-based absorbent article, further comprising a pair of back ears joined to the back region of the chassis region, and a pair of front ears joined to the front region of the chassis region.

In some embodiments, the bio-based absorbent further comprises a fastening system for joining the front and back regions to each other.

For the purposes of the present application, the following terms shall have the following meanings:

As used herein, the term “absorbent article” refers to devices which absorb and contain body exudates, and, more specifically, refers to devices which are placed against or in proximity to the body of the wearer to absorb and contain the various exudates discharged from the body.

The term “disposable” is used herein to describe absorbent articles which are not intended to be laundered or otherwise restored or reused as an absorbent article (i.e., they are intended to be discarded after a single use and, preferably, to be recycled, composted or otherwise disposed of in an environmentally compatible manner).

A “composite” absorbent article refers to absorbent articles which are formed of separate parts united together to form a coordinated entity so that they do not require separate manipulative parts like a separate holder and liner.

As used herein, the term “diaper” refers to an absorbent article generally worn by infants and incontinent persons that is worn about the lower torso of the wearer. It should be understood, however, that the present invention is also applicable to other absorbent articles such as incontinent briefs, incontinent undergarments, diaper holders and liners, feminine hygiene garments, and the like.

As used herein, the term “bio-based material” or “bio-based materials” refers to a material derived from natural processes such as agriculture, forestry, or other biological materials that are renewed or replenished to remain available for future generations. Bio-based materials can thus be contrasted with petroleum sourced material, such as synthetic polymers where the supply of petroleum is not naturally replenished in a reasonable length of time. Bio-based polymers are derived from a bio-based material. Bio-based carbon content can be verified with the help of a C-14 method according to ASTM D-6866 thus providing a route to verification of the bio-based content to the end use consumer.

The term “fiber” can refer to a fiber of finite length or a filament of infinite length.

As used herein the term “nonwoven web” means a structure or a web of material which has been formed without use of weaving or knitting processes to produce a structure of individual fibers or threads which are intermeshed, but not in an identifiable, repeating manner. Nonwoven webs have been, in the past, formed by a variety of conventional processes such as, for example, meltblown processes, spunbond processes, and staple fiber carding processes.

As used herein, the term “meltblown” refers to a process in which fibers are formed by extruding a molten thermoplastic material through a plurality of fine, usually circular, die capillaries into a high velocity gas (e.g. air) stream which attenuates the molten thermoplastic material and forms fibers, which can be to microfiber diameter, such as less than 10 microns in diameter. Thereafter, the meltblown fibers are carried by the gas stream and are deposited on a collecting surface to form a web of random meltblown fibers. Such a process is disclosed, for example, in U.S. Pat. No. 3,849,241 to Butin, et al.; U.S. Pat. No. 4,307,143 to Meitner, et al.; and U.S. Pat. No. 4,707,398 to Wisneski, et al., which are incorporated herein in their entirety by reference. Meltblown fibers in accordance with embodiments of the present invention may have circular and non-circular cross sections.

As used herein, the term “spunbond” refers to a process involving extruding a molten thermoplastic material as filaments from a plurality of fine, usually circular, capillaries of a spinneret, with the filaments then being attenuated and drawn mechanically or pneumatically. Based on the configuration of the spinneret orifice, fibers of various cross-section shapes can be produced including circular and non-circular, such as tri-lobal, delta, and the like shaped fibers. The filaments are deposited on a collecting surface to form a web of randomly arranged substantially continuous filaments which can thereafter be bonded together to form a coherent nonwoven fabric. The production of spunbond non-woven webs is illustrated in patents such as, for example, U.S. Pat. Nos. 3,338,992; 3,692,613, 3,802,817; 4,405,297 and 5,665,300. In general, these spunbond processes include extruding the filaments from a spinneret, quenching the filaments with a flow of air to hasten the solidification of the molten filaments, attenuating the filaments by applying a draw tension, either by pneumatically entraining the filaments in an air stream or mechanically by wrapping them around mechanical draw rolls, depositing the drawn filaments onto a collection surface to form a web, and bonding the web of loose filaments into a nonwoven fabric. The bonding can be any thermal or chemical bonding treatment, with thermal point bonding being typical. Other methods such a mechanical and hydroentanglement may also be used.

As used herein “thermal point bonding” involves passing a material such as two or more webs of fibers to be bonded between a heated calender roll and an anvil roll. The calender roll is typically patterned so that the fabric is bonded in discrete point bond sites rather than being bonded across its entire surface.

As used herein the term “polymer” generally includes, but is not limited to, homopolymers, copolymers, such as, for example, block, graft, random and alternating copolymers, terpolymers, etc. and blends and modifications thereof. Furthermore, unless otherwise specifically limited, the term “polymer” shall include all possible geometrical configurations of the material, including isotactic, syndiotactic and random symmetries.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is an illustration of an absorbent article in accordance with at least one embodiment of the present invention;

FIG. 2 is cross-sectional view of the absorbent article of FIG. 1 taken along line 2-2 of FIG. 1;

FIG. 3 is an illustration of an absorbent article in accordance with at least one embodiment of the present invention; and

FIG. 4 is an illustration of an absorbent article in accordance with at least one embodiment of the present invention in which the absorbent article is in the form of a feminine sanitary pad.

### DETAILED DESCRIPTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Embodiments of the present invention are directed to absorbent articles having a high bio-based material content. Preferably, absorbent articles in accordance with the embodiments of the present invention have a bio-based material content of at least 75 weight % of the absorbent article, such as comprising a bio-based material content that is at least 80%, 85%, 90%, or 95% by weight of the absorbent article.

In some embodiments, the bio-based material may comprise bio-based or biodegradable polymer materials. “Biodegradable” refers to a material or product which degrades or decomposes under environmental conditions that include the action of microrganisms. Thus a material is considered as biodegradable if a specified reduction of tensile strength and/or of peak elongation of the material or other critical physical or mechanical property is observed after exposure to a defined biological environment for a defined time. Depending on the defined biological conditions, a product comprised of a bio-based material might or might not be considered biodegradable.

A special class of biodegradable product made with a bio-based material might be considered as compostable if it can be degraded in a composing environment. The European standard EN 13432, “Proof of Compostability of Plastic Products” may be used to determine if a fabric or film comprised of sustainable content could be classified as compostable.

In one aspect, embodiments of the invention are directed to absorbent articles comprising a topsheet, a backsheet, and an absorbent core sandwiched therebetween. Preferably, the topsheet and backsheet are attached to each other along opposing surfaces to define a cavity in which the absorbent core is enclosed. As noted above, the components of absorbent article are selected so that at least 75% of the material comprising the absorbent article comprises a bio-based material, and preferably at least 80%, 85%, 90%, and 95% of the material comprising the absorbent article comprises a bio-based material.

Preferably, the absorbent article is substantially free of synthetic materials, such as petroleum-based materials and polymers. For example, absorbent articles in accordance with the present invention have less than 25 weight percent of materials that are non-bio-based, and more preferably, less than 20 weight percent, less than 15 weight percent, less than 10 weight percent, and even more preferably, less than 5 weight percent of non-bio-based materials, based on the total weight of the absorbent article.

Examples of absorbent articles in accordance with embodiments of the present invention include pant type absorbent articles, such as diapers, pull-ups, sanitary pants, and incontinence pants, and feminine absorbent articles, such as sanitary napkins. In one embodiment, the present invention is directed to disposable absorbent articles, such as disposable diapers.

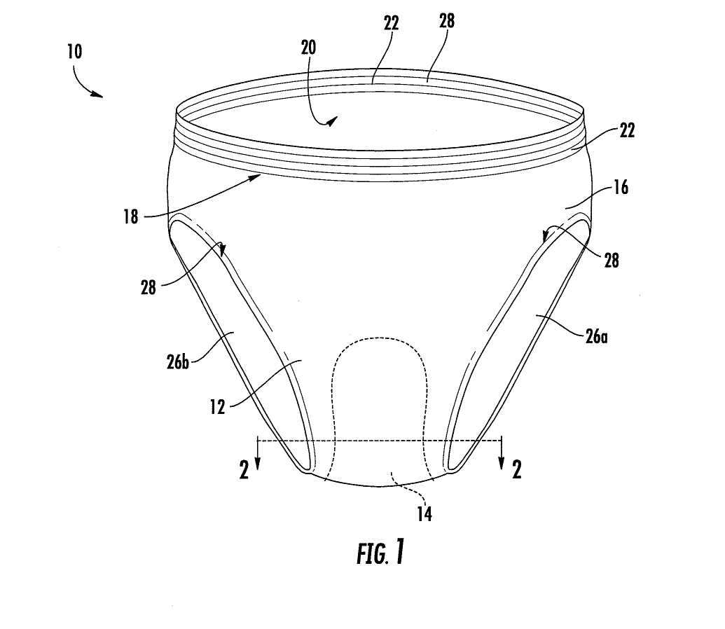

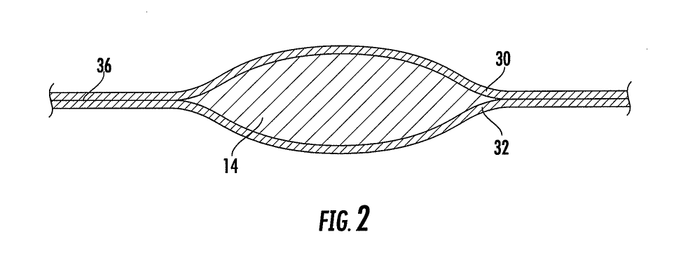

With reference to FIGS. 1-2, a pant type absorbent article (referred to herein simply as a “diaper”) in accordance with an embodiment of the present invention is illustrated and broadly designated by reference character 10. The diaper 10 includes a core region 12 in which an absorbent core 14 is disposed. A chassis region 16 surrounds the core region 12. The chassis region includes a front 18, back 20, and waist regions 22. The core region 12 is generally positioned in the crotch area of the diaper and extends at least partially into the front 18 and back 20 regions of the diaper.

The diaper shown in FIG. 1 is generally intended to enclose the lower part of the wearer's trunk like a pair of absorbent pants. As shown, the diaper may include leg openings 26a, 26b through which the wearer's legs are inserted. Although not illustrated in the embodiment of FIG. 1, the diaper may also include elastic cuffs that are disposed about the perimeter of the leg openings in order to contain fluids or exudates within the diaper.

In some embodiments, the diaper may also include elastic elements 28 that are disposed around one or more of the waist region 22 and leg openings 26a, 26b. The elastic elements may comprise elastic strings or threads that are contractably affixed between the topsheet and backsheet of the diaper.

In other embodiments, the front and back regions of the diaper may be joined to each other along adjacent longitudinal edges with ultrasonic, thermal, adhesive seals, or the like.

The chassis region comprised of front, back and core regions generally has a composite structure comprising a liquid permeable topsheet and a liquid impermeable backsheet that are attached to each other along opposing surfaces to define a cavity therebetween in which the absorbent core is disposed. In this regard, FIG. 2 shows a cross-section of the diaper taken along line 2-2 of FIG. 1 showing the absorbent core 14 sandwiched between the topsheet 30 and backsheet 32.

Topsheet

The topsheet 30 is positioned adjacent an outer surface of the absorbent core 14 and is preferably joined thereto and to the backsheet 32 by attachment means (not shown) such as those well known in the art. For example, the topsheet 30 may be secured to the absorbent core 14 by a uniform continuous layer of adhesive, a patterned layer of adhesive, or an array of separate lines, spirals, or spots of adhesive

As used herein, the term “joined” encompasses configurations whereby an element is directly secured to the other element by affixing the element directly to the other element, and configurations whereby the element is indirectly secured to the other element by affixing the element to intermediate member(s) which in turn are affixed to the other element. In a preferred embodiment of the present invention, the topsheet 30 and the backsheet 32 are joined directly to each other in the diaper periphery 36 and are indirectly joined together by directly joining them to the absorbent core 14 by the attachment means (not shown).

Preferably, the topsheet 30 is compliant, soft feeling, and non-irritating to the wearer's skin. Further, the topsheet 30 is liquid pervious permitting liquids (e.g., urine) to readily penetrate through its thickness. A suitable topsheet may be manufactured from a wide range of materials, such as porous foams; reticulated foams; apertured plastic films; or woven or nonwoven webs of natural fibers (e.g., wood or cotton fibers), or a combination of natural and synthetic fibers.

In one embodiment, the topsheet 30 is made of a hydrophobic material to help isolate the wearer's skin from liquids contained in the absorbent core 14.

In some embodiments, the topsheet may be treated with a surfactant to help ensure proper liquid transport through the topsheet and into the absorbent core. An example of a suitable surfactant is available from Momentive Performance Materials under the tradename NUWET™ 237.

Preferably, the topsheet comprises at least 75 weight percent of bio-based materials. Nonlimiting examples of bio-based polymers include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose; polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 11, alkyd resins, succinic acid-based polyesters, and bio-polyethylene terephthalate.

In a preferred embodiment, the bio-based polymers include polylactic acid and bio-based derived polyethylene. Generally, polylactic acid based polymers are prepared from dextrose, a source of sugar, derived from field corn. In North America corn is used since it is the most economical source of plant starch for ultimate conversion to sugar. However, it should be recognized that dextrose can be derived from sources other than corn. Sugar is converted to lactic acid or a lactic acid derivative via fermentation through the use of microorganisms. Thus besides corn other agricultural based sugar source could be used including sugar beets, sugar cane, wheat, cellulosic materials, such as xylose recovered from wood pulping, and the like. Similarly, bio-based polyethylene can be prepared from sugars that are fermented to produce ethanol, which in turn is dehydrated to provide ethylene.

In one embodiment, the topsheet 30 may comprise a nonwoven web comprising spunbond bicomponent fibers in which the fibers have a sheath-core configuration. In a preferred embodiment, the topsheet comprises spunbond bicomponent fibers having a core comprised of corn based polylactic acid (PLA), and a sheath comprising a sugar cane derived polyethylene polymer thus providing a topsheet of nearly 100% bio-based content. For use as a topsheet, such fabrics may desirably be treated with a surfactant such as suggested above to provide a hydrophilic surface. Nonwoven fabrics comprising PLA and bio-based polyethylene with basis weights of 13.5 GSM and 17 GSM, respectively, Grades 040RXEO09P and 050RXEO09P, surfactant treated to achieve a hydrophilic surface, are available from Fitesa Nonwovens of Simpsonville, S.C., 29681 USA.

An example of a suitable PLA polymer for the fiber core in such spunbond bicomponent fabrics is available from NatureWorks under the product name PLA Grade 6202. An example of a suitable sugar cane derived polyethylene is available from Braskem S.A. under the product name PE SHA7260. Advantageously, a topsheet comprising spunbond bicomponent fibers having a PLA core and a sheath comprising a sugar cane derived polyethylene polymer provides mechanical strength from the PLA core and improved softness from the polyethylene sheath.

In another embodiment, the polyethylene sheath of the bicomponent fiber may be replaced with a petroleum based polypropylene polymer to provide a topsheet with 50% bio-based content. Preferred polypropylenes for use in this embodiment will typically have a melt flow rate (MFR) between 20 to 40 g/10 min (measured in accordance with ASTM D1238 (190° C./2.16 kg)) such as for example provided by Total Petrochemicals and Refining USA, Inc. of La Port, Tex., 77571 USA as grades M 3766 (metallocene polypropylene) and 3764 or 3866 (Zeigler Natta polypropylene). Such Nonwovens, comprised of PP/PLA and showing 50% bio-based content, are available with basis weights of 13.5 GSM and 17 GSM as Grades 04PXBO09P and 050PXBG09P, respectively from Fitesa Nonwovens of Simpsonville, S.C., 29681 USA.

Further examples of nonwoven fabrics which after surfactant treatment can be used as topsheet in accordance with embodiments of the present invention include nonwoven webs, providing 50% bio-based content, comprising spunbond bicomponent fibers in which the core comprises a lignin based polymer and a sheath comprising a petroleum based polyethylene. Examples of such fabrics are disclosed as examples 4, 5, 6, 7, 8, and 9 in European Patent No. EP 2,630,285 B1 and U.S. Patent Publication No. 2014/0087618. Substitution of the petroleum based polyethylene sheath in these examples with a sheath comprised of either the sugar cane derived polyethylene available from Braskem S.A. or the corn derived PLA available from NatureWorks, both polymers disclosed above, would provide topsheets of nearly 100% bio-based content.

A further example of a topsheet that may be used comprises a surfactant treated nonwoven web comprising spunbond bicomponent fibers having a core of (PLA), and a sheath comprising PLA. For example, in one embodiment, the core may comprise a PLA having a lower % D isomer of polylactic acid than that of the % D isomer PLA polymer used in the sheath. The PLA polymer with lower % D isomer will show higher degree of stress induced crystallization during spinning while the PLA polymer with higher D % isomer will retain a more amorphous state during spinning. The more amorphous sheath will promote bonding while the core showing a higher degree of crystallization will provide strength to the fiber and thus to the final bonded web. In one particular embodiment, the Nature Works PLA Grade PLA 6752 with 4% D Isomer can be used as the sheath while NatureWorks Grade 6202 with 2% D Isomer can be used as the core.

A further example of a nonwoven fabric that could be used as a topsheet in accordance with embodiments of this invention may include thermobonded carded webs comprised of cotton and polypropylene. Depending on the fibers employed such webs may or may not require addition of surfactants (as described above) to achieve a desired hydrophilic nature for use as topsheet nonwovens. Examples of polypropylene staple fibers useful to form such fabrics are available from Fibervisions Corporation as Grade T-198. Examples of cotton fibers for use to form such nonwoven fabrics include fibers sold under the product name TRUECOTTON® available from TJ Beall Company, and fibers sold under the product name HIGH-Q ULTRA® available from Barnhardt Manufacturing Company.

Preferably, the topsheet has a basis weight from about 8 to about 25 grams per square meter, and more preferably from about 12 to 17 grams per square meter.

There are a number of manufacturing techniques which may be used to manufacture the topsheet 30. For example, the topsheet 30 may be a nonwoven web of fibers. When the topsheet comprises a nonwoven web, the web may be spunbonded, carded, wet-laid, meltblown, hydroentangled, combinations of the above, or the like. A preferred topsheet comprises a spunbond nonwoven fabric in which the fibers are thermally bonded to each other to form a coherent web.

Backsheet

The backsheet 32 is positioned adjacent to an opposite surface of the absorbent core 14 and is preferably joined thereto by attachment mechanisms (not shown) such as those well known in the art. Suitable attachment mechanisms are described with respect to joining the topsheet 30 to the absorbent core 14. Alternatively, the attachment means may comprise heat bonds, pressure bonds, ultrasonic bonds, dynamic mechanical bonds, or any other suitable attachment means or combinations of these attachment mechanisms as are known in the art.

The backsheet 32 is impervious to liquids (e.g., urine) and is preferably manufactured from a thin plastic film, although other flexible liquid impervious materials may also be used. As used herein, the term “flexible” refers to materials which are compliant and will readily conform to the general shape and contours of the human body. The backsheet 32 prevents the exudates absorbed and contained in the absorbent core 14 from wetting articles which contact the diaper 10 such as bedsheets and undergarments. The backsheet 32 may thus comprise a woven or nonwoven material, polymeric films such as thermoplastic films, or composite materials such as a film-coated nonwoven material. Preferably, the backsheet is a thermoplastic film comprising a high content of bio-based materials. The backsheet may have a thickness of from about 0.012 mm (0.5 mil) to about 0.051 mm (2.0 mils).

Material for the backsheet may include the bio-based polymers discussed previously. For example, bio-based polymers for use in the backsheet may include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose; polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 11, alkyd resins, succinic acid-based polyesters, and bio-polyethylene terephthalate.

In one embodiment the backsheet can be a film comprised of bio-based polymers as previously discussed. In one example the film may be comprised of low density polyethylene (LDPE) derived from sugar cane such as provided by grades SEB853 or SLL118/21 available from Braskem S.A.

In one embodiment, the backsheet may comprise a laminate structure having a liquid impervious film layer that is joined to a nonwoven web. Suitable films may be prepared from the bio-based polymers as previously discussed. In one example, the film may comprise a sugar cane derived polyethylene polymer, such as a film grade LDPE polyethylene grade SEB853/72 or SPB681/59 recommended by Braskem S.A. for lamination. Suitable films may also include additives such as CaCO₃ to improve film breathability while still maintaining fluid barrier properties.

In one embodiment, nonwovens for use in a laminated backsheet may include bio-based nonwovens as discussed above in connection with the topsheet. In one embodiment, such nonwovens may have a basis weight from 8 to 25 g/m². For backsheet lamination, such nonwovens will preferably be made without application of surfactants. For example, the nonwoven web may comprise spunbond bicomponent fibers in which the fibers have a sheath-core configuration where the sheath and/or the core can be comprised of such bio-based polymers as polyethylene from sugar cane, PLA from corn, or lignin recovered from wood pulp manufacture for paper.

In some embodiments, the backsheet layer may comprise a laminate structure having a bio-based film layer, such as those discussed previously, that is laminated to a fabric layer having a spunbond-meltblown-spunbond (SMS) structure. In such embodiments, the meltblown layer may typically have a basis weight ranging from 1 to 3 g/m², and the spunbond layers will typically have a basis weight ranging from 8 to 25 g/m². Suitable bio-based materials for the meltblown and spunbond layers are discussed above.

The size of the backsheet 32 is generally dictated by the size of the absorbent core 14 and the exact diaper design selected. For example, in some embodiments the backsheet 32 has a modified hourglass shape extending beyond the absorbent core 14 a minimum distance of at least about 1.3 cm to about 2.5 cm (about 0.5 to about 1.0 inch) around the entire diaper periphery.

Absorbent Core

The absorbent core 14 may comprise any material that is capable of absorbing fluids and exudates. Preferably, the absorbent core comprises at least 75% by weight of bio-based materials. In the past suitable materials for use as the absorbent core included pulp, such as cellulosic pulp, tissue layers, and fluff pulp. However the trend toward thin diapers has required the replacement of increasing pulp content with synthetic superabsorbent polymers such as superabsorbent polymers available from BASF sold under the trademark of HYSORB®. Use of increasing quantities of such superabsorbent polymer has significantly reduced the weight % content of sustainable content in current thin diapers sold in Western Europe and the USA.

A preferred embodiment of the diaper of this invention is comprised of superabsorbent polymers comprised of monomers of significant bio-based material content. Use of bio-based acrylic acid monomer is an example of a route to bio-based superabsorbent polymer that may be used in embodiments of the invention. Sugar from corn is converted to 3-hydroxypropionic acid (3-HP) which is then converted to glacial acrylic acid. The resulting bio-based glacial acrylic acid is used to make bio-based superabsorbent polymers. In one embodiment, the absorbent core may comprise a bio-based superabsorbent polymer derived from glacial acrylic acid. Such bio-based superabsorbent polymers have been developed by BASF, Cargill, and Novozymes. A superabsorbent polymer comprising up to 90 weight % bio-based sourced polyacrylic acid is discussed in U.S. Patent Publication No. 2013/0274697.

The absorbent core 14 may be manufactured in a wide variety of sizes and shapes (e.g., rectangular, hourglass, “T”-shaped, asymmetric, etc.). The configuration and construction of the absorbent core may also be varied (e.g., the absorbent core may have varying caliper zones, a hydrophilic gradient, a superabsorbent gradient, or lower average density and lower average basis weight acquisition zones; or may comprise one or more layers or structures). The total absorbent capacity of the absorbent core 14 should, however, be compatible with the design loading and the intended use of the diaper 10. Further, the size and absorbent capacity of the absorbent core 14 may be varied to accommodate wearers ranging from infants through adults.

In some embodiments, the absorbent core may include a fluid acquisition distribution system. This system may comprise an acquisition layer, which is placed adjacent to or in proximity of topsheet 30. The acquisition layer helps to distribute fluids along the absorbent core to help improve efficiency, and to reduce or prevent fluid leakage. When present, the acquisition layer may comprise a bio-based material based acquisition layer (AQL layer). In one embodiment, an AQLL may be made by carding a web comprised of a blend of 7 denier hollow PLA—Type 820 2 inch cut length staple fibers (Fiber Innovations Technology—Johnson City, Tenn.) plus 3 denier Solid PLA—Type 821 2 inch length staple fibers (Fiber Innovations Technology—Johnson City, Tenn.); treating the resulting web via a kiss roll with a suspension of cooked starch (Type STABITEX 65401 from Cargill); exposing the resulting web of fiber and starch to elevated temperature via a combination of hot air and contact to steam heated dryer cans to both cure and dry the web; and winding the resulting web into a roll and slitting the resulting roll into children rolls. The resulting AQL layer fabric may be comprised of nearly 100% bio-based material content.

In some embodiments, the distribution system may also include a distribution layer that is disposed underneath the acquisition layer. In some embodiments, the distribution layer may be a core cover or core wrap that covers or surrounds the absorbent material of the absorbent core to prevent particles of the absorbent core from contacting the baby's skin. The function of typical core wrap is discussed in U.S. Pat. No. 5,458,592. Preferably, the core wrap permits fluid to pass into the absorbent core while maintaining containment of the absorbent material. In one embodiment, materials for the core wrap may comprise a fabric layer comprising a spunbond fabric, spunbond-meltblown fabric (SM), or an SMS fabric. Suitable bio-based materials for the spunbond and meltblown layers of the core wrap are discussed above in connection with the topsheet.

One example of a core wrap comprising an SM fabric or SMS fabric comprises one or more spunbond nonwoven layers comprising bicomponent fibers having a polypropylene sheath and a PLA core, which is joined to a layer comprising polypropylene meltblown fibers.

Another example of a core wrap comprising an SMS fabric comprises a spunbond nonwoven layer comprising bicomponent fibers having a PLA sheath (e.g., Nature Works PLA Grade PLA 6752 with 4% D Isomer), and a PLA core (e.g. NatureWorks Grade 6202 with 2% D Isomer). In one embodiment, the meltblown layer of the SMS fabric may be comprised of a PLA meltblown fibers (e.g., NatureWorks PLA grade 6252).

In a third embodiment, the core wrap may comprise an SMS fabric in which the the spunbond nonwoven layers comprise biocomponent fibers comprising a polypropylene sheath, and a PLA core. Examples of suitable poplypropylenes typically have a melt flow rate (MFR) between 20 to 40 g/10 min (measured in accordance with ASTM D1238 (190° C./2.16 kg)) such as for example provided by Total Petrochemicals and Refining USA, Inc. of La Port, Tex., 77571 USA as grades M 3766 (metallocene polypropylene) and 3764 or 3866 (Zeigler Natta polypropylene). Suitable materials for the PLA core are available from NatureWorks, such as under the product name PLA Grade 6202.

In one embodiment, the meltblown layer for use in the SMS fabrics may comprise meltblown fibers comprising a blend of PLA and polypropylene that has been reclaimed from spunbond bicomponent fibers comprised of PP/PLA using the process taught in the international Application PCT/US 2015/012658, the contents of which are hereby incorporated by reference. Such meltblown webs are generally compatible for bonding to the sheath of the above bicomponent spunbond layers to provide a high bio-based material content SMS core wrap.

Typically, the spunbond bicomponent webs for use in the core wrap, have a sheath/core ratio that may be from approximately 30/70 to 70/30. For the above examples total basis weigh of the resulting SMS may be between about 8 g/m² and about 15 g/m² with the meltblown content being approximately 10% of the weight, based on the total weight of the fabric.

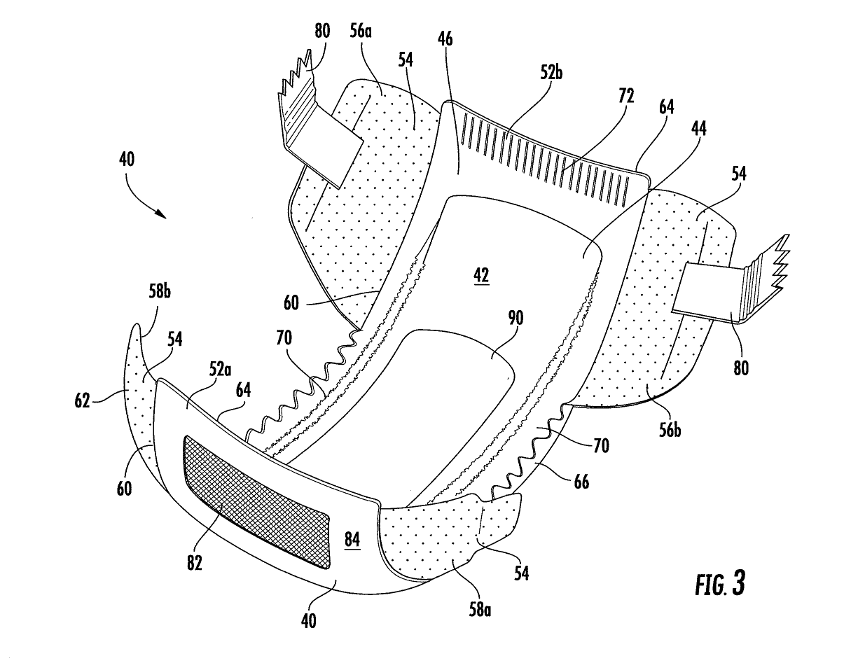

With reference to FIG. 3, another embodiment of an absorbent article (“diaper”) in accordance with embodiments of the present invention is shown and broadly designated by reference number 40. As in the embodiment discussed previously, the diaper 40 includes a core region 42 in which an absorbent core 44 is disposed. A chassis region 46 surrounds the core region 42, and includes a front 48, back 50, and front and back waist regions 52a, 52b. The chassis region comprised of front, back and core regions generally has a composite structure comprising a liquid permeable topsheet and a liquid impermeable backsheet that are attached to each other along opposing surfaces to define a cavity there between in which the absorbent core is disposed.

Suitable materials for the topsheet, backsheet, and absorbent core are discussed previously.

In a preferred embodiment, the front and back regions of the diaper also each includes a pair of ears 54 that are disposed in the waist regions of the diaper. (As used herein, the term “disposed” is used to mean that an element(s) of the diaper is formed (joined and positioned) in a particular place or position as a unitary structure with other elements of the diaper or as a separate element joined to another element of the diaper.) The ears 54 provide an elastically extensible feature that provides a more comfortable and contouring fit by initially conformably fitting the diaper to the wearer and sustaining this fit throughout the time of wear well past when the diaper has been loaded with exudates since the elasticized side panels allow the sides of the diaper to expand and contract.

In addition, the ears 54 develop and maintain wearing forces (tensions) that enhance the tensions developed and maintained by a fastening system, discussed in greater detail below, to maintain the diaper 40 on the wearer and enhance the waist fit. As shown in FIG. 3, the diaper includes a pair of back ears 56a, 56b which are joined to the back region 50 of the diaper chassis proximate to the back waist region 52b, and a pair of front ears 58a, 58b, which are joined to the front region 48 of the diaper chassis proximate of the front waist region 52a.

The front and back ears may be joined to the chassis region 46 by any bonding method known in the art such as adhesive bonding, pressure bonding, heat bonding, and the like. In other embodiments, the front and/or back ears may comprise a discrete element joined to the chassis region with the chassis region 46 having a layer, element, or substrate that extends over the front and/or back ear. For example, each ear may comprise a portion of the diaper chassis region that extends laterally outwardly from and along the side edge 60 of the chassis region to a longitudinal edge 62 of the diaper 40. In one embodiment, the ears generally extend longitudinally from the end edge 64 of the diaper 40 to the portion of the longitudinal edge 62 of the diaper 20 that forms the leg opening (this segment of the longitudinal edge 62 being designated as leg edge 66). In some embodiments, the ears may comprise a separate fabric or web that has been joined to the topsheet or the backsheet. In other embodiments, each ear may be formed by the portions of the topsheet and the backsheet that extend beyond the side edges of the absorbent core 44.

The front ears and back ears may be extensible, inextensible, elastic, or inelastic. The front ears and back ears may be formed from nonwoven webs, woven webs, knitted fabrics, polymeric and elastomeric films, apertured films, sponges, foams, scrims, and combinations and laminates thereof. In certain embodiments the front ears and back ears may be formed of a stretch laminate comprising a first nonwoven, elastomeric material, and, optionally, a second nonwoven or other like laminates. In a preferred embodiment, front and back ears comprise nonwovens that are derived from a bio-based material. A suitable elastomeric material may comprise a natural elastomer such as natural rubber.

Preferably, the ears are comprised of at least 75% by weight of a bio-based material. In a preferred embodiment, the ear flaps comprise a laminate material. In one embodiment, the ears comprise an elastic material. In other embodiments, the ears comprise an extensible material. The ears can be integral part of the chassis, for example formed from the topsheet and/or backsheet as a side panel. Alternatively, they may be separate elements attached by gluing and/or heat embossing or pressure bonding. In some embodiments, the back ears are advantageously stretchable to facilitate the attachment of tabs 80 on a landing zone 82, and to maintain the taped diapers in place around the wearer's waist. The back ears may also be elastic or extensible to provide a more comfortable and contouring fit by initially conformably fitting the absorbent article to the wearer and sustaining this fit throughout the time of wear well past when absorbent article has been loaded with exudates since the elasticized ears allow the sides of the absorbent article to expand and contract.

In one embodiment, the back ears may comprises a laminate comprising a three layer structure in which an elastic film is disposed between two nonwoven layers. The two nonwoven layers may be the same or different from each other.

Nonwoven fabrics for lamination with the elastic film to provide the above described back ears must balance two conflicting properties: sufficiently high cross-directional extensibility to allow mechanical activation of the elastic film fabric laminate to provide elasticity to insure proper fit, and a sufficiently directional stability (low neck-in under machine direction tension) to make both the nonwoven and the resulting laminate processable on high speed converting lines. Examples of nonwovens offering such balanced and conflicting properties are taught and claimed in European Patent No. EP 2524077 B1 and U.S. Patent Publication No. 2014/0072788. Laminates comprised of such nonwovens are taught and claimed in U.S. Pat. No. 8,728,051, the contents of which are hereby incorporated by reference.

Fabrics and laminates for use in preferred embodiments of the back ears may have the added challenge of being comprised of bio-based material content. In one embodiment, the nonwoven for lamination to the elastic film comprises spunbond bicomponent fibers in which the fibers have a sheath-core configuration such that the sheath is comprised of a sugar cane derived polyethylene polymer. An example of a suitable sugar cane derived polyethylene is available from Braskem S.A. under the product name PE SHA7260, and the core is comprised of petroleum based polypropylene where preferred polypropylenes for use in this embodiment will typically have a melt flow rate (MFR) between 20 to 40 g/10 min (measured in accordance with ASTM D1238 (190° C./2.16 kg)) such as for example provided by Total Petrochemicals and Refining USA, Inc. of La Port, Tex., 77571 USA as grades M 3766 (metallocene polypropylene) and 3764 or 3866 (Zeigler Natta polypropylene).

A nonwoven for a particularly preferred embodiment is comprised of bicomponent sheath/core spunbond fiber fabric where the sheath is comprised of the above bio-base derived polyethylene, such as sugar can derived polyethylene (available from Braskem S.A.) and the core is comprised of Total M 3766 polypropylene where the nonwoven is processed by generally following the procedure outlined for Examples 1, 2 and 3 of U.S. Patent Publication No. 2014/0072788. The above nonwoven fabric may be, for example, laminated to an elastic film, ring rolled and then incorporated into an absorbent article via current art high speed diaper converting steps of attachment to provide an absorbent article with stretchable back ears such that the absorbent article provides leak free fit and comfort to the wearer.

In a preferred embodiment, the front ears may comprise a spunbond fabric or an SMS fabric. Suitable materials for forming the front ears may comprise bio-based materials discussed above in connection with the topsheet, backsheet, core wrap, or leg cuffs. Preferably, the front ears comprises a fabric layer having a basis weight ranging from about 25 to 50 g/m².

In one embodiment, the diaper 40 may also include elastic leg cuffs 70 for providing improved containment of fluids and other body exudates. Each elasticized leg cuff 70 may comprise several different embodiments for reducing the leakage of body exudates in the leg regions. (The leg cuff can be and is sometimes also referred to as leg bands, side flaps, barrier cuffs, or elastic cuffs.) U.S. Pat. No. 3,860,003 entitled “Contractable Side Portions for a Disposable Diaper” issued to Buell on Jan. 14, 1975, describes a disposable diaper which provides a contractible leg opening having a side flap and one or more elastic members to provide an elasticized leg cuff (gasketing cuff). U.S. Pat. No. 4,909,803 entitled “Disposable Absorbent Article Having Elasticized Flaps” issued to Aziz and Blaney on Mar. 20, 1990, describes a disposable diaper having “stand-up” elasticized flaps (barrier cuffs) to improve the containment of the leg regions. U.S. Pat. No. 4,695,278 entitled “Absorbent Article Having Dual Cuffs” issued to Lawson on Sep. 22, 1987, describes a disposable diaper having dual cuffs including a gasketing cuff and a barrier cuff. U.S. Pat. No. 4,704,115 entitled “Disposable Waist Containment Garment” issued to Buell on Nov. 3, 1987, discloses a disposable diaper or incontinent garment having side-edge-leakage-guard gutters configured to contain free liquids within the garment. Each of these patents are incorporated herein by reference. U.S. Pat. No. 6,476,289 entitled “Garment Having Elastomeric Laminate” describes various elastic leg cuff configurations that may also be used in embodiments of the present invention.

In a preferred embodiment, the leg cuffs may comprises a fabric layer having an SMS structure comprising a plurality of elastic strands that are incorporated into the leg cuff structure. Preferably, the leg cuffs comprises a material having liquid barrier properties.

One example of a fabric for use in forming leg cuffs comprises an SMS fabric having a spunbond nonwoven layer comprising bicomponent fibers having a polypropylene sheath and a PLA core. An example of a polypropylene material for use in this embodiment may have a melt flow rate (MFR) between 20 to 40 g/10 min (measured in accordance with ASTM D1238 (190° C./2.16 kg)) such as, for example, provided by Total Petrochemicals and Refining USA, Inc. of La Port, Tex., 77571 USA as grades M 3766 (metallocene polypropylene) and 3764 or 3866 (Zeigler Natta polypropylene). A suitable material for use as the PLA core is available from Nature Works PLA as Grade 6202 with 2% D Isomer. The meltblown layer may comprise a polypropylene having an MFR of 1,300 g/10 min (measured in accordance with ASTM D1238 (190° C./2.16 kg)) such as, for example, provided by Total Petrochemicals and Refining USA, Inc. of La Port, Tex., 77571 USA as grade 3962.

In a second example, the leg cuffs may comprise an SMS fabric having a spunbond nonwoven layer comprising bicomponent fibers having a PLA sheath and a PLA core, and a meltblown layer comprising PLA fibers. An example of a suitable PLA material for use as the sheath is PLA grade 6752 with 4% D Isomer, and an example of a suitable PLA material for use as the core is PLA grade 6202 with 2% D Isomer, both of which are available from NatureWorks. A suitable material for the PLA meltblown fibers is PLA grade 6252, which is also available from NatureWorks.

In a third embodiment, the leg cuffs may comprise a fabric having an SMS structure in which the spunbond nonwoven layers comprise a bicomponent fabric having a polypropylene sheath and a PLA core. Examples of suitable materials for the sheath and core are described above. The meltblown layer may comprise meltblown fibers comprising a blend of PLA and polypropylene that has been reclaimed from spunbond bicomponent fibers comprised of PP/PLA using the process taught in International Application PCT/US 2015/012658.

In a fourth embodiment, the leg cuffs may comprise a fabric having an SMS structure in which the spunbond nonwoven layers comprise a bicomponent fabric having a PLA sheath and a PLA core. Examples of suitable materials for the sheath and core are described above. As in the third embodiment discussed above, the meltblown layer may comprise meltblown fibers comprising a blend of PLA and polypropylene that has been reclaimed from spunbond bicomponent fibers comprised of PP/PLA using the process taught in International Application No. PCT/US2015/012658.

Preferably, spunbond fabrics for forming the leg cuffs have a sheath/core ratio of approximately 30/70 to 70/30. In one embodiment, the basis weight of the SMS fabric is between about 8 g/m² and 15 g/m². Preferably, the meltblown content comprises about 10 to 30 weight %, based on the total weight of the SMS fabric. In some embodiments, the SMS fabric for use in forming the leg cuffs has a hydrohead value of greater than about 50 mm as measured in accordance with INDA Test Method WSP 80.6.

As in the previously discussed embodiment, the diaper 40 may also include elastic elements that are disposed around one or more of the waist region 52 and the elastic cuffs. For example, the diaper may also comprise at least one elastic waist feature (not represented) that helps to provide improved fit and containment. The elastic waist feature is generally intended to elastically expand and contract to dynamically fit the wearer's waist. The elastic waist feature preferably extends at least longitudinally outwardly from at least one waist edge of the absorbent core and generally forms at least a portion of the end edge of the absorbent article. Disposable diapers can be constructed so as to have two elastic waist features, one positioned in the front waist region and one positioned in the back waist region. The elastic waist feature may be constructed in a number of different configurations including those described in U.S. Pat. No. 4,515,595, U.S. Pat. No. 4,710,189, U.S. Pat. No. 5,151,092 and U.S. Pat. No. 5,221,274.

In some embodiments, the elastic features may comprise elastic elements comprising elastic strands or threads that are contractably affixed between the topsheet and backsheet of the diaper. Such strands or threads can be comprised of a bio-based material, such as natural rubber. As noted above the natural rubber strands are covered by nonwoven, such as the topsheet and/or backsheet to insure elastic component does not directly contact the wearer's skin.

The absorbent article may include a fastening system. The fastening system can be used to provide lateral tensions about the circumference of the absorbent article to hold the absorbent article on the wearer as is typical for taped diapers. This fastening system is not necessary for pull on style of absorbent articles, such as training pants or adult incontinence absorbent articles, since the waist region of these articles is already bonded.

The fastening system usually comprises a fastener such as tape tabs, hook and loop fastening components, interlocking fasteners such as tabs & slots, buckles, buttons, snaps, and/or hermaphroditic fastening components, although any other known fastening means are generally acceptable. A landing zone is normally provided on the front waist region for the fastener to be releasably attached. When fastened, the fastening system interconnects the front waist region 52a and the back waist region 52b. When fastened, the diaper 44 contains a circumscribing waist opening and two circumscribing leg openings.

The fastening system may comprise an engaging member 80 and a receiving member 82 (also referred to as a landing zone). The engaging member 80 may comprise hooks, loops, an adhesive, a cohesive, a tab, or other fastening mechanism. The receiving member 82 may comprise hooks, loops, a slot, an adhesive, a cohesive, or other fastening mechanism that can receive the engaging member 80. Suitable engaging member 80 and receiving member 82 combinations are well known in the art and include but are not limited to hooks/loop, hooks/hooks, adhesive/polymeric film, cohesive/cohesive, adhesive/adhesive, tab/slot, and button/button hole. Suitably, the fastening system may comprise a polymer derived from a bio-based material.

In this regard, FIG. 3 shows a fastening system in which the engaging member comprises a pair of tabs 80 that are joined to the back ears 56a, 56b, and an associated landing zone 82 disposed on a front surface 84 of the diaper 40. In some embodiments, the tabs may include a pressure sensitive adhesive for adhesively attaching the tabs to the landing zone.

Some exemplary surface fastening systems are disclosed in U.S. Pat. No. 3,848,594, U.S. Pat. No. 4,662,875, U.S. Pat. No. 4,846,815, U.S. Pat. No. 4,894,060, U.S. Pat. No. 4,946,527, U.S. Pat. No. 5,151,092 and U.S. Pat. No. 5,221,274 issued to Buell. An exemplary interlocking fastening system is disclosed in U.S. Pat. No. 6,432,098. The fastening system may also provide a means for holding the article in a disposal configuration as disclosed in U.S. Pat. No. 4,963,140 issued to Robertson et al.

The fastening system may also include primary and secondary fastening systems, as disclosed in U.S. Pat. No. 4,699,622 to reduce shifting of overlapped portions or to improve fit as disclosed in U.S. Pat. No. 5,242,436, U.S. Pat. No. 5,499,978, U.S. Pat. No. 5,507,736, and U.S. Pat. No. 5,591,152.

In a preferred embodiment, the fastening system can employ a hook and loop as described in U.S. Pat. No. 9,084,701 where both the hook and the loop components are comprised of significant bio-based material content. In a preferred embodiment, the hook and loop fastening system comprises a female fastening material made of fibrous material and a male fastening material with hooks configured for the fibrous material.

In one embodiment, the female loop material comprises bonded bicomponent fibers comprising a bio-based material, such as spunbond bicomponent fibers having a PLA, and a sheath comprising a sugar cane derived polyethylene polymer. Examples of such materials are described above. An example of a suitable PLA polymer for the core in is available from Nature Works as PLA Grade 6202.

A second fiber for use as the female loop component providing 50% bio-based material content comprises a sheath of petroleum based polypropylene polymer and a PLA core derived from NatureWorks under the product name PLA Grade 6202. Preferred polypropylenes for use in this embodiment will typically have a melt flow rate (MFR) between 20 to 40 g/10 min (measured in accordance with ASTM D1238 (190° C./2.16 kg)) such as for example provided by Total Petrochemicals and Refining USA, Inc. of La Port, Tex., 77571 USA as grades M 3766 (metallocene polypropylene) and 3764 or 3866 (Zeigler Natta polypropylene).

A further example of fibers for constructing a female loop material, providing 50% bio-based material content, comprise spunbond bicomponent fibers in which the core comprises a lignin based polymer and a sheath comprising a petroleum based polyethylene. Such fibers are disclosed as examples 4, 5, 6, 7, 8, and 9 in European Patent No. EP 2,630,285 B1 and U.S. Patent Publication No. 2014/0087618.

Substitution of the petroleum based polyethylene sheath in these examples with a sheath comprised of either the sugar cane derived polyethylene available from Braskem S.A. or the corn derived PLA available from NatureWorks, both polymers disclosed above, would provide fibers having up to a 100% bio-based material content.

A further example of a fiber that can be used for constructing the female loop material is a bicomponent fiber having a core of (PLA), and a sheath comprising PLA. For example, in one embodiment, the core may comprise a PLA having a lower % D isomer of polylactic acid than that of the % D isomer PLA polymer used in the sheath. The PLA polymer with lower % D isomer will show higher degree of stress induced crystallization during spinning while the PLA polymer with higher D % isomer will retain a more amorphous state during spinning. The more amorphous sheath will promote bonding will the core showing a higher degree of crystallization will provide straight to the fiber and thus to the final bonded web.

In one particular embodiment, the Nature Works PLA Grade PLA 6752 with 4% D Isomer can be used as the sheath while NatureWorks Grade 6202 with 2% D Isomer can be used as the core.

A further example of fibers for use in the female loop material, providing at least 50 bio-based material content may comprise a 50/50 blend of cotton fibers and a petroleum based polymer, such as polypropylene. Examples of polypropylene staple fibers useful to form such fabrics are available from Fibervisions Corporation as Grade T-198. Examples of cotton fibers for use to form such nonwoven fabrics include fibers sold under the product name TRUECOTTON® available from TJ Beall Company, and fibers sold under the product name HIGH-Q ULTRA® available from Barnhardt Manufacturing Company.

The male hooks use in this fastening stem for the preferred embodiment are also comprised of significant sustainable content. The male fastening material including the hooks can be made by casting, molding, profile extrusion, or microreplications where the polymer used is corn derived PLA such as is available from NatureWorks. NatureWorks provides a selection of grades for injection molding that could be used to make such hooks including Grades 3001D, 3052D, 3100HP and 3251D.

In some embodiments, the diaper may also include a fluid acquisition distribution system. In this regard, FIG. 3 includes a system having at least a fluid acquisition layer 90. As discussed above, the acquisition layer helps to efficiently transfer fluid to the absorbent core 44. Fabrics useful as acquisition layer are discussed above.

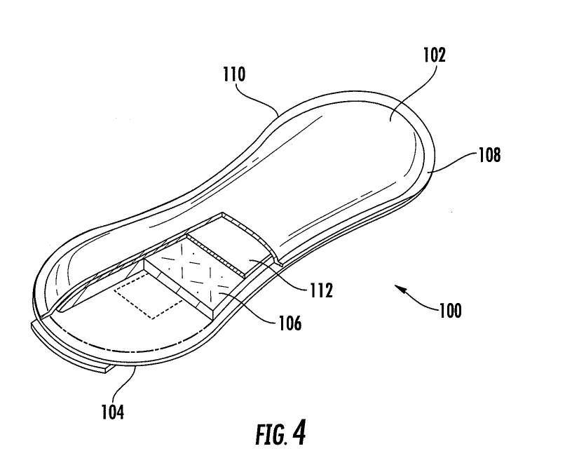

With reference to FIG. 4, a further embodiment of an absorbent article in accordance with an embodiment of the present invention is illustrated in which the absorbent article is in the form of a feminine sanitary pad, broadly designated by reference character 100.

Pad 100 may include a topsheet 102, backsheet 104, and an absorbent core 106 disposed there between. Preferably, topsheet 102 and backsheet 104 are joined to each other about along opposing outer edges to define a continuous seam 108 that extends about the periphery 110 of the pad 100. Continuous seam 108 may comprise a heat seal that is formed from thermally bonding the topsheet and backsheet to each other. In other embodiments, continuous seam 108 is formed by adhesively bonding the topsheet and backsheet to each other. Preferably, the adhesive is a bio-based adhesive as discussed previously.

As in the embodiments discussed above, pad 100 comprises a bio-based material content of at least 75 weight percent, based on the total weight of the pad, such as comprising a bio-based material content that is at least 80%, 85%, 90%, or 95% by weight of the pad.

Suitable materials for the topsheet, backsheet, and absorbent core are discussed previously

In some embodiments, pad 100 may also include a fluid acquisition layer 112 that is disposed between the absorbent core 106 and the topsheet 102. Suitable materials for the fluid acquisition layer 112 are discussed previously.

Various components of the absorbent article are typically joined via thermal or adhesive bonding. When an adhesive is employed, the adhesive preferably comprises a bio-based adhesive. An example of a bio-based adhesive is a pressure sensitive adhesive available from Danimer Scientific under the product code 92721.

In the above examples, PLA is generally discussed in terms of being derived from corn. However, one of ordinary skill in the art would recognize that PLA polymers may be derived from other bio-based materials that are capable of being converted to lactic acid. Examples of materials for producing PLA may include sugar beets, sugar cane, wheat, cellulosic materials, such as xylose recovered from wood pulping, and the like. Similarly, bio-based polyethylene is discussed in terms of being derived from sugar cane. One of ordinary skill in the art would recognize that bio-based polyethylene may derived from any material, such as sugar, that can be fermented to produce ethanol. Examples of such materials may include both biological and agricultural sources, such as those noted above, bacteria, yeast, corn, cellulose based materials, and the like.

Although the absorbent articles have generally been discussed in terms of bio-based content, it should be understood that the absorbent article may also include non-bio-based materials. For example, non-bio based polymers that may be used in the invention include polyethylene's, polypropylenes, polyesters, nylons, synthetic rubbers, and homopolymers and copolymers thereof.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A bio-based absorbent article comprising a topsheet, a backsheet, and an absorbent core disposed there between, wherein the absorbent article has a bio-based material content of at least 75 weight percent, based on total weight of the absorbent article.

2. The bio-based absorbent article of claim 1, wherein one or more of the topsheet and backsheet comprises a nonwoven web comprising spunbond bicomponent fibers in which the fibers have a sheath-core configuration.

3. The bio-based absorbent article of claim 1, wherein the fibers having a core of polylactic acid (PLA), and a sheath comprising a bio-based derived polyethylene polymer.

4. The bio-based absorbent article of claim 3, wherein the fibers having a core of polylactic acid (PLA), and a sheath comprising a polypropylene polymer.

5. The bio-based absorbent article of claim 3, wherein the fibers having a core of a lignin based polymer and a sheath comprising a bio-based derived polyethylene.

6. The bio-based absorbent article of claim 3, wherein the fibers comprise a core of (PLA), and a sheath comprising PLA, wherein the core has a melting temperature that is higher than the melting temperature of the PLA polymer comprising the sheath.

7. The bio-based absorbent article of claim 1, wherein the topsheet and backsheet are adhesively joined to each other with a bio-based adhesive.

8. The bio-based absorbent article of claim 1, wherein the absorbent core comprises an acrylic polymer that is derived from the conversion of bio-based 3-hydroxypropionic acid (3-HP) to glacial acrylic acid.

9. The bio-based absorbent article of claim 1, wherein the backsheet comprises a laminate comprising a film layer of a sugar cane derived polyethylene polymer that is adhesively laminated to nonwoven web comprising spunbond bicomponent fibers in which the fibers have a sheath-core configuration.

10. The bio-based absorbent article of claim 9, wherein the fibers of the nonwoven web comprises 1) a core comprising polylactic acid (PLA), and a sheath comprising a bio-based derived polyethylene polymer, 2) fibers comprising a core of polylactic acid (PLA), and a sheath comprising a polypropylene polymer, 3) fibers having a core of a lignin based polymer and a sheath comprising bio-based derived polyethylene, or 4) fibers comprising a core of (PLA), and a sheath comprising PLA, wherein the core has a melting temperature that is higher than the melting temperature of the PLA polymer comprising the sheath.

11. The bio-based absorbent article of claim 1, wherein the article is in the form of a diaper.

12. The bio-based absorbent article of claim 1, wherein the article is in the form of a feminine sanitary pad.

13. The bio-based absorbent article of claim 1, wherein the absorbent article has a bio-based material content of at least 90 weight percent, based on total weight of the absorbent article.

14. A bio-based absorbent article, comprising a core region having an absorbent core and a chassis region surrounding the core region, said chassis region comprising front, back and waist regions, while the core region is located at least in a crotch portion of the article, a liquid impermeable backsheet is arranged at least in the core region on the garment-facing side of the absorbent core and a liquid permeable topsheet is arranged at least in the core region on the wearer-facing side of the absorbent core, wherein the absorbent article has a bio-based material content of at least 75 weight percent, based on total weight of the absorbent article.

15. The bio-based absorbent article of claim 14, further comprising a pair of back ears joined to the back region of the chassis region, and a pair of front ears joined to the front region of the chassis region.

16. The bio-based absorbent article of claim 14, further comprising a fastening system for joining the front and back regions to each other.

17. The bio-based absorbent article of claim 14, wherein the absorbent core comprises an absorbent material having a bio-based material content of at least 90 weight percent, based on total weight of the absorbent core.

18. The bio-based 7 absorbent article of claim 14, wherein the absorbent core comprises an acrylic polymer that is derived from the conversion of bio-based 3-hydroxypropionic acid (3-HP) to glacial acrylic acid.

19. The bio-based absorbent article of claim 14, wherein the backsheet comprises a laminate comprising a film layer of a bio-based derived polyethylene polymer that is adhesively laminated to nonwoven web comprising spunbond bicomponent fibers in which the fibers have a sheath-core configuration.

20. The bio-based absorbent article of claim 19, wherein the fibers of the nonwoven web comprises 1) a core comprising polylactic acid (PLA), and a sheath comprising a bio-based derived polyethylene polymer, 2) fibers comprising a core of polylactic acid (PLA), and a sheath comprising a polypropylene polymer, 3) fibers having a core of a lignin based polymer and a sheath comprising bio-based derived polyethylene, or 4) fibers comprising a core of (PLA), and a sheath comprising PLA, wherein the core has a melting temperature that is higher than the melting temperature of the PLA polymer comprising the sheath.

21. The bio-based absorbent article of claim 14, wherein the wherein the topsheet comprises a spunbond bicomponent fibers in which the fibers have a sheath-core configuration comprising: 1) a core comprising polylactic acid (PLA), and a sheath comprising a sugar cane derived polyethylene polymer, 2) fibers comprising a core of polylactic acid (PLA), and a sheath comprising a polypropylene polymer, 3) fibers having a core of a lignin based polymer and a sheath comprising sugar cane derived polyethylene, or 4) fibers comprising a core of (PLA), and a sheath comprising PLA, wherein the core has a melting temperature that is higher than the melting temperature of the PLA polymer comprising the sheath.

22. The bio-based absorbent article of claim 14, wherein the absorbent article has a bio-based material content of at least 90 weight percent, based on total weight of the absorbent article.

23. The bio-based absorbent article of claim 14, further comprising a leg cuff attached to the chassis region of the article, the leg cuff comprising a spunbond-meltblown-spunbond fabric layer, wherein the spunbond and meltblowns comprise PLA.
